(19) United States
(12) Patent Application Publication
(10) Pub. No.: US 2023/0354477 A1
(43) Pub. Date: Nov. 2, 2023

(54) HEATING DEVICE FOR DISPENSING VOLATILE COMPOUNDS AND METHOD OF USE

(71) Applicant: Scott Outdoors, Rehobeth, AL (US)

(72) Inventors: James Scott TURNER, Rehobeth, AL (US); Michael Scott DEAL, Eufaula, AL (US); Terry Lance BEASLEY, Dothan, AL (US)

(21) Appl. No.: 18/137,068

(22) Filed: Apr. 20, 2023

Related U.S. Application Data
(60) Provisional application No. 63/335,814, filed on Apr. 28, 2022.

(51) Int. Cl. H05B 1/02; A61M 11/04

(52) U.S. Cl. CPC H05B 1/0294; A61M 11/042; H05B 2203/02; H05B 2203/003; A61M 2205/3368; A61M 2205/8206

(57) ABSTRACT

A heating device for volatizing materials contained in an impregnated pad has a housing including a chamber to receive an impregnated pad for heating, a battery contained in the housing, and a heater arrangement. The heater arrangement in one mode includes at least two heaters, a temperature sensor, and a controller, wherein the controller is configured to power the first heater based on a first temperature and then to power the second heater after the first temperature reaches a second temperature. In another mode, the heater arrangement has at least one heater, temperature sensor, and controller, wherein the controller is configured to provide battery power to the at least one heater to volatize materials in the impregnated pad. The housing has at least one opening to allow volatized materials from the impregnated pad to escape from the housing.

### FIELD OF THE INVENTION

The present invention relates to heating devices for dispensing volatile compounds, such as from pads impregnated with scented compounds and methods of use.

### BACKGROUND ART

Volatile compound-impregnated pads composed of, for example, compressed wood fibers are a commonly used delivery system for scented compounds, natural and artificial pesticides and insect repellents, wildlife attractants, etc. The compounds are dispersed from the impregnated pad into the air by means of a heating device powered by gas flame or electrochemical battery. Though gas-powered flame heaters have primary market success, they have significant drawbacks, chiefly 1) cost of the gas canisters, 2) poor cold weather performance (the gas canisters loose pressure), and 3) lack of fixed temperature control.

### SUMMARY OF THE INVENTION

In one aspect, the invention provides a significant improvement in the way impregnated pads are heated for purposes of volatilizing the compounds impregnated in the pads for a desired use, one being to release scents in the pads to attract game for hunting, and another to release insect repellents and insecticides. More particularly, the invention provides an improved heating device that is battery-powered and works well in cold environments that would normally be problematic in terms of battery drain. This improvement relates to at least a dual heating element arrangement and a controller that enables energizing one of the heating elements until a desired temperature is reached and the other heating element can then be energized for maximum heating of the impregnated pad. Energizing one of the heating elements first allows the other heating element temperature to rise before it is energized so as to avoid a drain on the battery if both heating elements were energized at the same time in a low temperature environment.

In another aspect, the invention provides a significant improvement in safety for electrochemical powered heating devices by employing multiple, independent means to detect when a heating device has been left on and stowed. A heating device can present a significant fire hazard if stowed for example, along with other common items in a backpack or satchel. In such an enclosed environment, heat energy from the device will accumulate given the insulating nature of the backpack and its contents. This heat can reach temperatures exceeding 500° F., above the ignition temperature of common items. This can be worsened if live ammunition is found among the stowed items.

The invention also has means for detecting an unsafe condition by detecting an abnormally low compensating power, together with a rising ambient temperature and providing the ability to shut down the heater. The heating device can also account for windy conditions and adjust the temperature of the heating device based on such conditions.

In yet another embodiment of the invention, the heating device enjoys the synergy of a number of important features: 1) A single circuit board with integral heating element, controls, battery connections and heat loss prevention features, 2) an enclosure designed to shield and insulate the heating element to prevent heat and thus power loss, and 3) a scent pad with integral insulating properties to further prevent heat loss.

In addition to those direct improvements, the invention enjoys minimal manufacturing costs as the printed circuit board comprises both the control circuit and heating elements, as opposed to incorporating a separately manufactured heating element with the attendant additional costs of handling and assembly.

In one embodiment of the invention, the heating device for volatizing materials contained in an impregnated pad includes a housing including a chamber to receive an impregnated pad for heating, a battery contained in the housing, and a heater arrangement. In one mode, the heater arrangement includes at least first and second heaters mounted on a substrate contained in the housing and in communication with the chamber to heat the impregnated pad, at least one temperature sensor; and a controller contained in the housing. Each of the first and second heaters comprise a printed circuit trace on the substrate.

The controller is provided to supply battery power to each of the at least two heaters, wherein the controller is configured to power the first heater based on a first temperature and then to power the second heater after the first temperature reaches a second temperature.

An alternative heater arrangement can be employed with the temperature sensor and controller, this arrangement including at least one heater mounted on the substrate contained in the housing and in communication with the chamber to heat the impregnated pad. For this arrangement, the controller provides battery power to the at least one heater to volatize materials in the impregnated pad.

The housing is configured to have at least one opening to allow volatized materials from the impregnated pad to escape from the housing.

The at least one opening in the housing can include a plurality of slits in a portion of the housing, the slits configured to minimize water entry through the slits.

For the first heater arrangement, the first temperature is that temperature at which the combined load of the at least first and second heaters does not exceed the battery's maximum output wattage.

The housing can also have access opening to allow removal and replacement of the battery.

The controller can be configured to shut down power to the device based on an overheating condition for the heating device.

While the housing can have any shape, the housing is preferably sized to be handheld.

For the heating arrangements, the controller can be configured to lower power to the at least two heating elements under windy conditions.

In another operating mode for the controller, wherein there are at least two heaters, the controller is configured to operate only one of the at least two heating elements for volatizing.

The invention also entails an improvement in methods wherein a device is used to heat an impregnated pad to volatize compounds impregnated in the pad. The improvement in this method is the heating the pad using the inventive heating device.

A more specific heating device includes at least first and second heaters mounted on a substrate in communication with each other, at least one temperature sensor, optionally more than one temperature sensor, and a controller providing battery power to each of the at least two heating elements. The controller is configured to power the first heater based on a first temperature, then to power the second heater after the first heating element raises the temperature of both heating elements to a second temperature. This heating device includes one or more of: a housing to house the at least first and second heaters, the at least one temperature sensor, and the controller; a battery; and a printed circuit board as the at least first and second heaters.

The housing can include a space adjacent to a surface of the substrate that is opposite to a surface of the substrate that faces the at least one opening, the space including insulation therein.

In the alternative heating arrangement, at least two heaters are provided, the at least two heaters are mounted on the substrate and each controlled individually.

While the substrate can have any number of configurations, a preferred on is where the substrate has a first portion for supporting either the first and second heaters or at least one heater and a second portion supporting the controller, the first and second portions separated by opposing slots forming a connecting portion to minimize heat conduction between the first and second portions.

In yet another embodiment, the housing includes slots sized to receive and hold peripheral ends of the substrate supporting the at least first and second heaters or the at least one heater to minimize heat conduction from the substrate to the housing.

### BRIEF DESCRIPTION OF THE DRAWINGS

FIG. 1 shows a schematic drawing of one embodiment of the invention and core components of the inventive heater.

FIG. 2 shows a graph showing the effect of temperature on current.

FIG. 3 shows a perspective view of a part of a housing of the heater with the heating element and controller therein.

FIG. 4 shows a portion of a housing with an insulating feature.

FIG. 5 shows a schematic of a portion of the heating device showing a heat loss feature.

FIG. 6 shows a schematic of a portion of the heating device showing another heat loss feature.

FIG. 7 shows a schematic of a portion of the heating device showing yet another heat loss feature.

FIGS. 8A and 8B show schematics of a portion of the heating device showing still another heat loss feature.

FIG. 9 shows a portion of the heating device with another heat loss feature.

FIG. 10 shows a schematic representation of the component parts making up the inventive heater.

FIG. 11 shows a schematic representation of the housing securing an impregnated pad in place.

FIG. 12 shows a front view of the housing and an exemplary louvered grill design for the heater.

FIG. 13 shows a sectional view of the louvered grill design of FIG. 12.

FIG. 14 shows another embodiment of the invention, using a different heater arrangement.

FIG. 15 shows another embodiment of the invention in terms of the configuration of the housing.

FIG. 16 shows the circled parts of FIG. 15 magnified to show more detail.

### DETAILED DESCRIPTION OF THE INVENTION

The inventive device includes, in one embodiment, a printed circuit board (PCB) preferably comprised of a control circuit, dual heater control elements (such as MOSFETs), heating elements composed of PCB traces, one or more temperature sensors (such as thermistors), preferably mounted among the heating traces, and a battery for power.

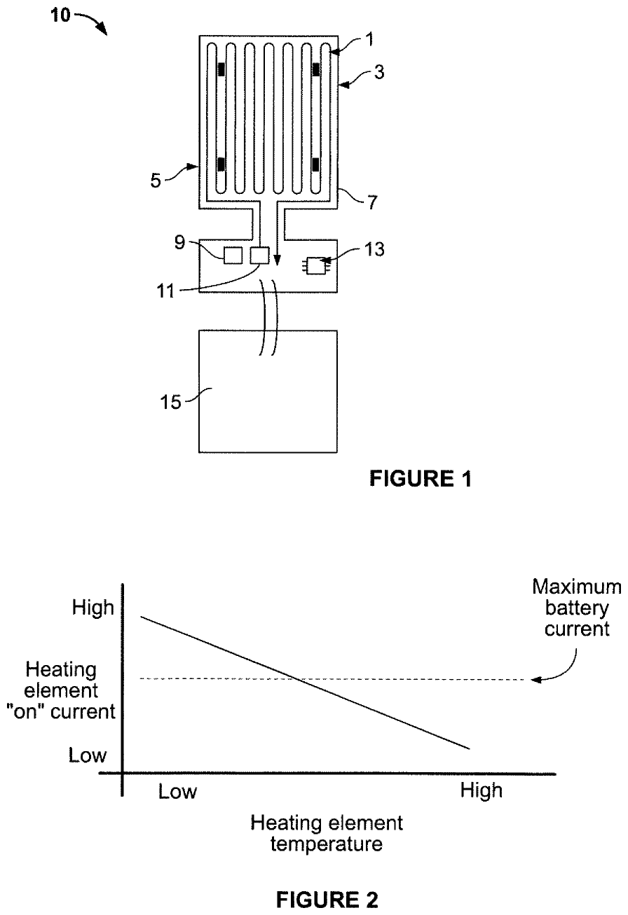

FIG. 1 shows an example of such a heating device 10 with top and bottom heating elements or traces 1 and 3, four temperature sensors 5 spaced amongst the traces 1 on one side of a substrate 7, a part of which functions as the board of the PCB to provide a base for the traces 1 and 3.

Also mounted to the substrate is a pair of control elements 9 and 11, one for the top or one side heating element 1 and one for the bottom or opposite side heating element 3. A microprocessor 13 is also provided as part of the controller of the device. The battery 15 is shown schematically connected to the control circuit. While not shown in FIG. 1, the component parts of the inventive heater are contained in a housing as described in more detail below. This design shown in FIG. 1 allows for an efficient manufacturing cost, and presents a significant improvement over the prior art heating devices.

It is also important that the heating elements present a current load less than the maximum allowed by the battery. This presents a major design problem if the heating element's temperature may start as low as, for example, 10° F., but may heat the scent pad to temperatures in excess of, for example, 280° F. The series resistance may increase by over 40% over this temperature range. Considering the batteries must be used at maximum load to drive the heating element to its maximum temperature, a cold heating element would present a load well beyond the maximum power available from the batteries, due to metals such as copper having a resistance with positive temperature coefficient. FIG. 2 illustrates the relationship between the heating current on versus the heating element temperature. At low temperatures, the heating current is high and above the maximum battery current and decreases as the heating element temperature increases. When using volatile oils in a scent pad in a battery powered heater, the heater must be heated to ˜280° F. This would require roughly an 8-watt heater against a 10° F. atmosphere. A PCB-based heating element that presents an 8 W load at 280° F., presents a 13.3 W load at 10° F. because of copper's decreasing resistance with decreasing temperature (aka positive temperature coefficient). Meaning an otherwise sufficient battery capable of delivering 1A into the 8 W load would have to supply 1.7A into the 13.3 W load. With the control scheme described above alleviates this problem.

The inventive heating control system more particularly solves this problem by employing at least two separate heating elements opposite each other, preferably on a two-sided printed circuit board. Of course, the two heating elements could be placed on one side of the PCB as well. Means are provided in terms of the control circuit to energize only one element of the two elements when the heating element is at low temperature. This allows both heating elements to be elevated to a temperature where their combined load would not exceed the battery's maximum current and both heating elements can be energized for heating the impregnated pad. An example of a temperature that the controller could be programmed for switching to powering both heating elements is 120° F. However, other temperatures can be used depending on the typical ambient temperatures where the heating device would be used. The temperature before turning on the second heater is a function of the heater's resistance vs temperature (i.e., thickness and width of the traces). Put another way, the switch-over temperature can be said to be that temperature at which the combined load of the at least two heaters does not exceed the battery's maximum output wattage, see FIG. 2.

The inventors anticipate other less efficient solutions to cold heating element compensation, such as co-planar intertwined heating elements and the temporary addition of resistance to the heating circuit with physical or virtual resistance (such as MOSFETs driven in the linear region). However, these solutions are less efficient as they dissipate heat either in an uneven manner, and/or in areas of the circuit where the heat (and thus battery capacity) would be wasted.

### Safety Feature

In one embodiment, the heating device can include a printed circuit board preferably comprised of a control circuit, dual heater control elements (such as MOSFETs), heating elements composed of PCB traces, temperature sensors (such as thermistors) mounted among the heating traces, the temperature sensors mounted to detect ambient temperature, and a battery for power. The heater control elements are driven preferably with a pulse width modulated (PWM) generated from the controller. The controller calculates the PWM on percentage based on feedback from the heating element temperature sensors. This comprises a closed-loop control system. It is important to note that any heater control circuit must employ a control method able to add additional power to compensate for the heat energy lost from the warm heating element into the atmosphere. The invention correlates the magnitude of this additional power with the ambient temperature.

If the heater is powered and stowed, the heater will begin to lose far less heat because of the insulating effect of the storage bag and contents. Consequently, the controller will adjust the output power lower and lower. The abnormally low heater control setting will trigger an automatic shutdown. This is one example of an overheating condition that requires the controller to take steps to avoid the overheating. Instead of an overheating condition, wherein the controller reacts to a lower and lower output power by an automatic shutdown as noted above, another overheating condition would be based on the temperature sensor(s) reaching a certain high temperature that would mandate a shutdown of battery power via the controller.

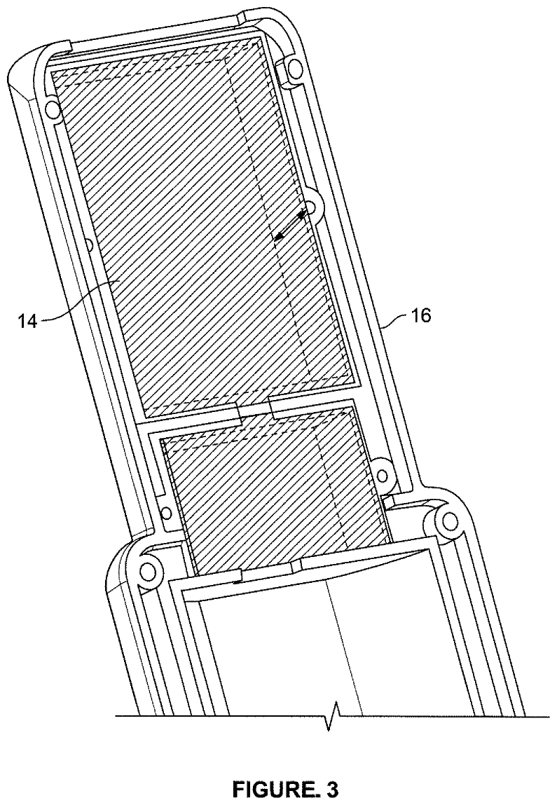

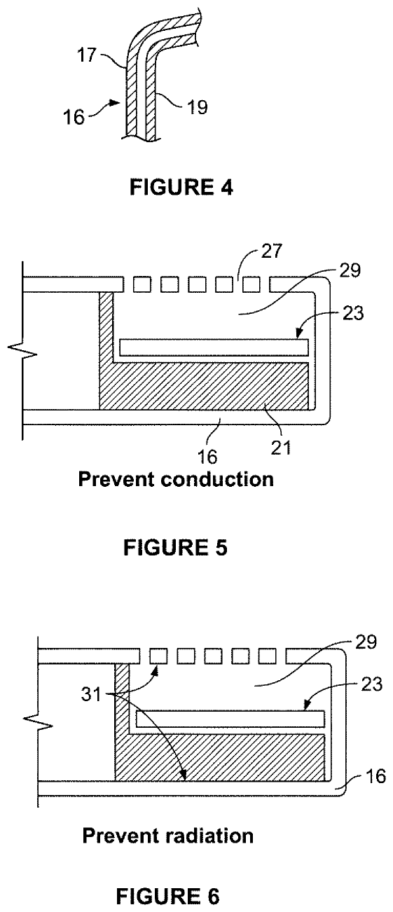

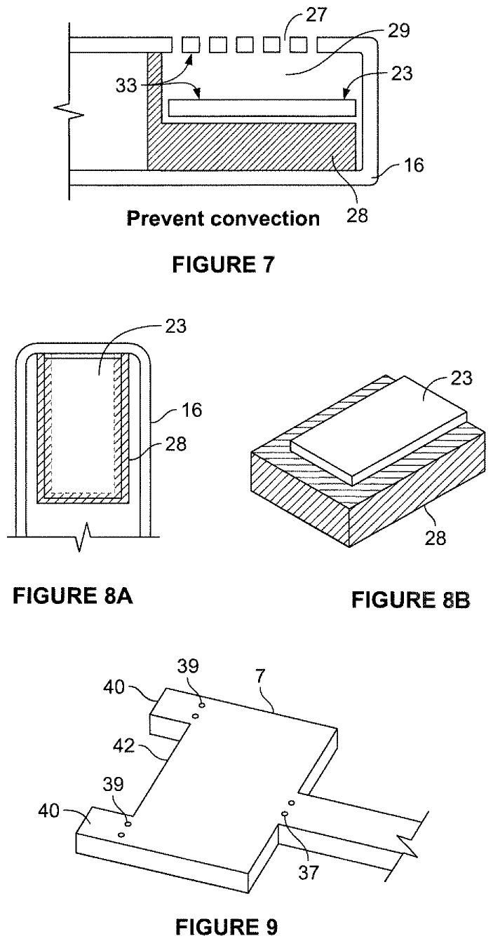

To improve the efficiency of the heating device, the device includes a number of different configurations as detailed below. FIG. 3 shows the PCB heating element 14 elevated from the housing 16. Referring to FIG. 4, a cross sectional corner portion of the housing is shown with a double wall construction 17, 19 to limit heat loss through conduction to the surface of the housing. Referring to FIG. 5, insulation 21 can be used beneath the heating elements on the PCB heating element 23 to prevent heat conduction through the bottom surface of the housing 16. FIG. 5 also shows locating the heating element 23 far from the exhaust grill 27 so as to create a vapor chamber 29. FIG. 6 shows the use of reflective material 31 to make the inside of the surfaces of the housing surrounding the heating element 23 reflective to infrared radiation. FIG. 7 employs a configuration wherein a large gap 33 is provided between the grill 27 and the top of the heating element 23 to reduce convection currents. The structure 28 in FIG. 7 that is below the heating element 23 is a perimeter support for the heating element. FIGS. 8A and 8B show the heating element 23 positioned in the housing 16 and on the peripheral support 28 from top and perspective views wherein most of the periphery of the heating element is exposed to prevent heat loss through the housing. FIG. 9 shows another feature of the invention in terms of minimizing heat loss. The substrate 7, shown without the traces, has a number of through openings 37 and 39. The openings 37 impede heat transfer towards the controller and the openings 39 impede heat transfer to the tabs 40, which are designed as spacers to limit contact between the entirety of the edge 42 of substrate and any parts of the housing facing the substrate edge.

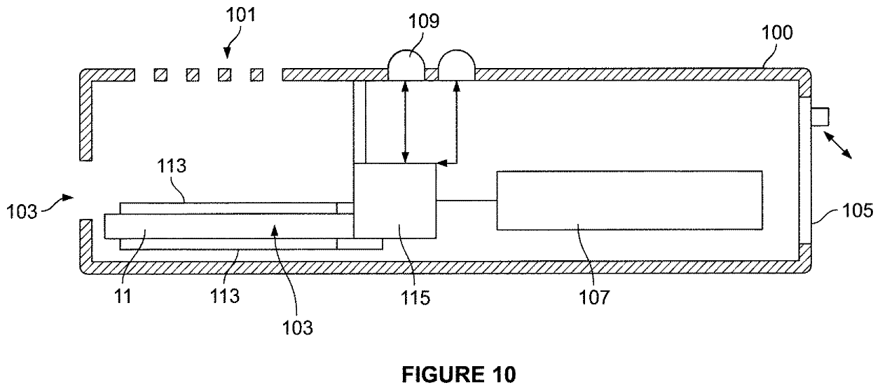

FIG. 10 shows a schematic representation of one embodiment of the inventive device. A housing is provided that encloses the battery, the controller, the heating element having the at least two traces on a substrate. While not shown, the temperature sensors could be on one or both sides of the substrate or located elsewhere in the housing providing the necessary temperature detection can be achieved to allow for proper control of the heating device. The housing 100 has a grill 101 to allow vapor from the impregnated pad (not shown) to be released into the atmosphere upon heating. The housing in this embodiment has a slot 103 to receive the pad (not shown), the pad placed on the heating element 103 for heating. The housing also includes an access door 105 to access the battery 107 held in the housing. An on off switch 109 is also depicted. A substrate 111 is shown with a heater trace 113 on either side, although only one side of the substrate could include the trace. The controller is shown as 115. It should be understood that any kind of on/off mechanism can be employed with the inventive heating device. The heating device could also have an external control that would allow setting a temperature for heating the pads. The heating device could also have wireless capability wherein the unit is controlled remotely using an app, tablet, or the like. Since this wireless control is well known, further description of its features is not necessary for understanding of the invention.

The manner of mounting the various components in the housing can also vary as well. For example, a single substrate could be used to mount the heating traces, controllers, and temperature sensors or the heating traces could be mounted separate from the controllers. The battery can be mounted in any way in the housing as well.

This novel design improves battery life and efficiency by minimizing heat loss through conduction through the sides of the enclosure, heat loss through conduction through the bottom of the enclosure, heat loss through radiation through the walls of the enclosure, and heat loss through convection.

The housing can have any type of access for the battery, e.g., a door, a removable cover portion, a slot that allows access for the battery to facilitate its removal and replacement.

The housing will also have any kind of an opening, which could include a removable cover or the slot as detailed above to allow the impregnated pad to be placed on the substrate for heating purposes. The pad could be designed with a gripping feature to allow the pad to be removed from the slot if a slot is used. The housing can have any shape to hold the component parts of the heating device. An elongated shape is preferred as the device will normally be hand held.

### Details of the Design

Preferably, the heating element's temperature can be optimally and precisely controlled by preferably using a proportional integral (PI), or proportional integral derivative (PID) temperature control method driving a pulse width modulation (PWM controller). As the heater may be accidentally stored in a concealed space (such as a backpack) a number of reliable means are employed to prevent overheating. While the PI/PID controller would hold the heating element at a constant temperature regardless of surroundings, significant heating could occur to flammable contents in contact with the heating element (such as clothing or live ammunition). The inventive heater would have means to detect such a condition in at least three different ways. A first way is by monitoring the ambient temperature. A second way is by cross comparing the ambient temperature to the PI/PID controllers output command. A third way is by monitoring the individual terms of the PI/PID loop control, such as for example, the integral term would be significantly smaller for a stowed heating element due to the additional insulation. The controlling firmware detects such dangerous conditions and automatically shuts down the device.

The inventive heating device can also use the PI/PID controller and the ambient temperature sensor as a means to detect wind conditions and adjust the heating device temperature under windy conditions. This is done by cross comparing the PWM command (and individual PI/PID terms) to the ambient temperature. A higher-than-normal PWM command for a given ambient temperature indicates windy conditions. The controller automatically reduces the heating pad temperature to compensate for the higher scent diffusion rate due to additional air flow.

The inventive heater is designed to be compatible with off-the-shelf rechargeable battery packs. However, these battery packs include a power supply that automatically powers down when no load is detected. As part of the inventive device, means are provided in terms of the controller operation to periodically cause power to be on briefly, for example every 15 seconds for a duration of 10 ms, to cause the battery pack controller to stay “on”, but in any event a period and duration suitable to the chosen model of battery pack to stay “on” while minimizing power usage. This prevents the user having to remove the battery pack from the invention to power it on manually. This “battery blip” feature makes the invention much easier to use.

Being battery-powered, the device should conserve the available energy in the battery to only heat the scent pad. Careful design of the enclosure affords longer battery life by allowing the liberation of volatile compounds from the impregnated pads, but with minimal wasted heat. It is important to realize that the volatile compounds are driven from the pad with heat, but once in the gaseous phase, those gases need not be heated.

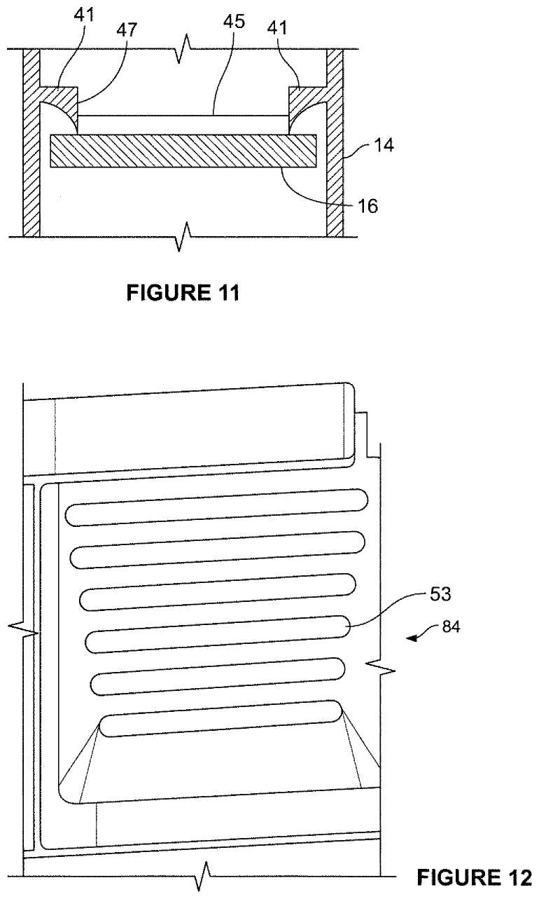

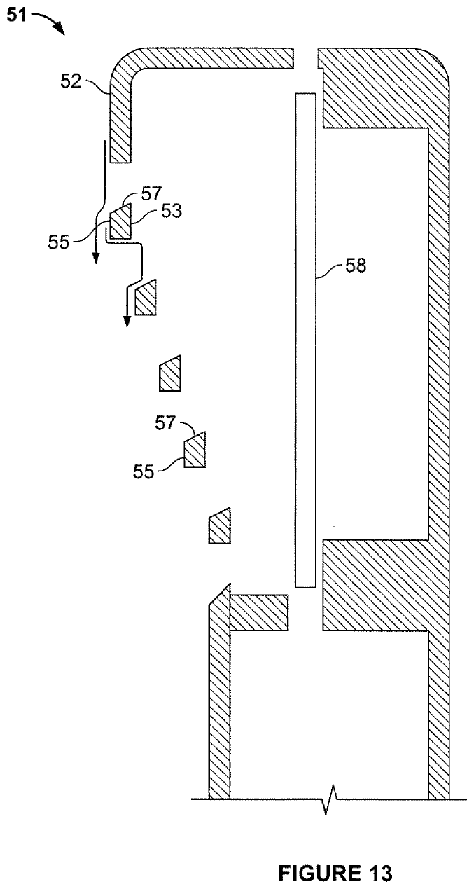

Thus, the inventive heating device employs extensive methods to channel all the heat energy into the scent pad using one or more of the following methods. Heat waste through conduction is limited such as with heat flow impeding perforations in the circuit board material, see FIG. 9, wherein the heat impedance perforations slow heat conduction to areas of the substrate other than those supporting the traces for heating purposes. The housing is also configured with guides designed to hold the pad in place on the substrate, the guides allowing minimal contact between the scent pad guides and heated PCB, see FIG. 11, wherein the guides 41 that are part of the housing and designed to guide the pad 45 into position on the heating element 16, would be configured such that only a small percentage area of the guides 41 rests on the heating element 16. In this embodiment, the guide has an edge that rests on the heating element 16, with a part of the adjacent face 47 facing the side edge of the pad. While not shown, the guides could be positioned intermittently and spaced apart along the side edges of the pad 45 or be configured as a one-piece guide that extends continuously along a length of the side edge of the pad. Heat loss through convection is also limited by design of the scent pad retaining grill to limit air flow across the scent pad, and FIG. 12 shows this particular grill design, which is designated by a louvered design 84 with spaced apart slats, the slats angling downward towards the scent pad. FIG. 13 shows a sectional view of the grill design of FIG. 12 in a hanging position such that the heating device would be vertically oriented. The arrows in FIG. 13 water flowing down the grill face 51 and surface 52. The grill slats 53 are designed with an outward facing surface 55 and another surface 57, which is angled with respect to the outward facing surface 55. With the heating device hanging from a support, water would run along face 52, then contact face 57 and be directed away from the heater 58 to the next outward facing surface 55 as shown by the arrow travel in FIG. 13. This design assists in keeping rain water away from the heater 58. The grill design shown in FIGS. 12 and 13 is one that can be easily molded as a single parting surface mold, thus reducing costs to make the heating device. Preventing water ingress to the heater and pad allows for longer operation in wet weather, directly improving marketability.

The inventive heating device takes advantage of the fact that a rechargeable battery's life is reduced in a non-linear fashion with increasing load. Thus, given the use of at least two separate heating elements, and the ability to be able to use only one heating element where possible, battery life is improved by limiting the peak current drawn by the PWM circuit. Where possible, the invention can also use one heating element at 100%, while using the second element sparingly. This also contributes to increased battery life. Of course, both heating elements could be used initially as well should conditions favor this mode of heating.

While various and different kinds of controller components are used, any known kinds of controllers can be used in connection with the heating device as means to accomplish the various functions described above, e.g., using just one heating element for pad heating, using both heating elements at one time, using the at least two heating elements in sequence, using the ambient temperature conditions and controller output for reducing the heating element temperature due to windy conditions, using ambient conditions and the controller output to monitor dangerous conditions and shut off the device.

While the heating device is primarily designed to heat scent pads for the purpose of vaporizing compounds that would attract game for hunting purposes, the pad could be any kind that contained a volatile compound whose vaporization provides some benefit, e.g., insect repellants, fragrances to provide a pleasant smell to a space, and the like.

Further yet, the heating device in terms of at least first and second heaters mounted on a substrate in communication with each other, at least one temperature sensor, optionally more than one temperature sensor, and a controller providing battery power to each of the at least two heating elements, wherein the controller is configured to power the first heater based on a first temperature, then to power the second heater after the first heating element raises the temperature of both heating elements to a second temperature, is one that can be used in any application that needs battery powered heat, and not just as a part of the scent pad heating device. In this embodiment, it is preferred to use the traces for the heating elements but other could be used. Moreover, any kind of housing could be used to house the heating device components. The heating device could also come with a battery or the battery could be provided independently of the heating device.

The grill design above could also be used in other applications where water ingress is undesirable, either for a heater or any other application where water is unwanted.

The insulating features described above could also be used in other housings that require some insulating capability.

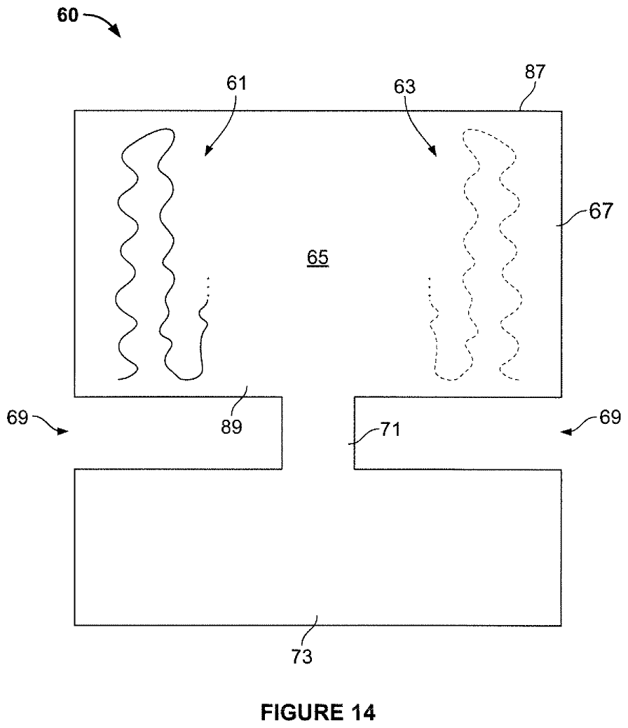

Another embodiment of the invention is depicted in FIGS. 14-16. In this embodiment and in contrast to the embodiment shown in FIG. 1 that required at least two heating elements and a specific control scheme, at least one single heating element is provided for volatilizing materials impregnated in a pad in this embodiment and the control scheme is much simpler.

A printed circuit board 60 that would be part of the heating device is depicted as having two heating elements 61 and 63, element 61 on surface 65 of the substrate 67 and element 63 on the other side of the substrate 67. A multi-layer circuit board would allow for multiple heating elements, each at different wattages. The substrate 67 is configured with a pair of slots 69 that form a connection portion 71. The purpose of the slots 69 that form the isthmus 71 is to maximize the separation of the heated portion of the substrate that holds the heating elements 61 and optionally 63 from the control circuit (not shown), which is located on portion 73 of the substrate 67. These slots 69 minimize heat conduction and also simplify sealing the control circuit from the moisture. While two heating elements are shown, only one could be used. Also, a temperature sensor (not shown for this embodiment) would also be employed, similar to that used in the FIG. 1 embodiment.

The surface 65 and heating element 61 are in contact with a scented pad and are thus exposed to the elements to facilitate release and dispersal of the volatile compounds. As such, surface 65 must be subjected to rain ingress, while the control circuit and element 67 (and co-located temperature sensing circuits) must be protected from water ingress. Thus, the perimeter of surface 65 must be sealed, preventing water from travelling from 65 to the control circuit, or to heating element 63 (and associated temperature sensing circuits co-located with 63). Slots 69 maximize the relatively simple perimeter-to-enclosure seal, leaving only isthmus 71 requiring a more complicated sandwich type seal.

In contrast to the control scheme used in the embodiment of FIG. 1, wherein one of the heating elements is used first, the control scheme for the embodiment of FIG. 14 is one wherein the control circuit, i.e., control element and microprocessor, located on the portion 73, and temperature sensor, would provide battery power to the heating element or elements based on a desired heating temperature for the volatile material on the scented pad.

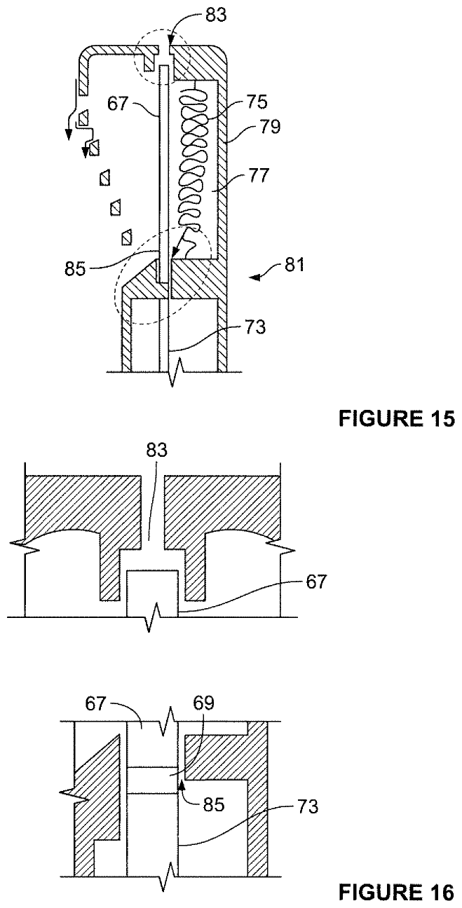

FIG. 15 and the closeup of the circled portions of FIG. 15 shown in FIG. 16 show further improvements to the housing configuration as depicted in FIG. 13. These improvements relate to a careful design of the housing to afford longer battery life and allowing the liberation of the volatiles of the pad but with minimal wasted heat. It should be noted that once the volatiles are released from the pad, and in the gaseous phase, there is no need to further heat the gases.

The housing employs a double-wall design like that described in connection with FIG. 4 to limit heat loss through conduction to the surface of the housing. Unlike FIG. 13, additional insulation 75 is included in the space between the substrate 61 and a portion 79 of the housing 81. This minimizes heat loss from the substrate to the portion 79 of the housing 81 that faces the substrate 61 and its heating elements. The rain diversion feature shown in FIG. 13 is also illustrated in FIG. 16.

FIG. 16 also shows a pair of slots 83 and 85 in the housing 81. These slots are minimally sized so that only the peripheral end 87 of the substrate, see FIG. 14, and the end 89 having the connecting portion extending therefrom rest in the slot. This configuration also minimizes heat conduction to the housing and leaves the maximum area of the substrate exposed for heating of the impregnated pad.

As such, an invention has been disclosed in terms of preferred embodiments thereof which fulfills each and every one of the objects of the present invention as set forth above and provides a new and improved device for heating impregnated pads to volatize scents therein and a method of use.

Of course, various changes, modifications and alterations from the teachings of the present invention may be contemplated by those skilled in the art without departing from the intended spirit and scope thereof. It is intended that the present invention only be limited by the terms of the appended claims.

1. A heating device for volatizing materials contained in an impregnated pad comprising:
a housing including a chamber to receive an impregnated pad for heating, a battery contained in the housing;
a heater arrangement comprising either:
a) at least first and second heaters mounted on a substrate contained in the housing and in communication with the chamber to heat the impregnated pad;
at least one temperature sensor; and
a controller contained in the housing;
each of the first and second heaters further comprise a printed circuit trace on the substrate;
the controller providing battery power to each of the at least two heaters, wherein the controller is configured to power the first heater based on a first temperature and then to power the second heater after the first temperature reaches a second temperature; or
b) at least one heater mounted on the substrate contained in the housing and in communication with the chamber to heat the impregnated pad,
at least one temperature sensor;
a controller contained in the housing;
wherein the at least one heater further comprises a printed circuit trace on the substrate;
the controller providing battery power to the at least one heater to volatize materials in the impregnated pad;
the housing having at least one opening to allow volatized materials from the impregnated pad to escape from the housing,
the battery connected to either the first and second heaters via the controller for supply of power to the first and second heaters or connected to the at least one heater via the controller for supply of power to the at least one heater.

2. The device of claim 1, wherein for heating arrangement (a), one printed circuit trace is on one side of the substrate and the other printed circuit trace is on the other side of the substrate.

3. The device of claim 1, wherein for heating arrangement (a), each printed circuit trace is on one side of the substrate.

4. The device of claim 1, wherein the at least one temperature sensor is mounted on the substrate.

5. The device of claim 1, wherein the substrate is mounted with the housing such that at least two sides of the substrate are spaced from the housing.

6. The device of claim 1, wherein inner surfaces of the housing facing the substrate include a reflective coating.

7. The device of claim 1, wherein a surface of the substrate is spaced from an underside of the portion of the housing forming the at least one opening.

8. The device of claim 1, wherein the at least one opening further comprises a plurality of slits in a portion of the housing, the slits configured to minimize water entry through the slits.

9. The device of claim 1, wherein the first temperature is that temperature at which the combined load of the at least first and second heaters does not exceed the battery's maximum output wattage.

10. The device of claim 1, wherein the housing has an access opening to allow removal and replacement of the battery.

11. The device of claim 1, wherein the controller is configured to shut down power to the device based on an overheating condition for the heating device.

12. The device of claim 1, wherein the housing is sized to be handheld.

13. The device of claim 1, wherein for heating arrangement (a), the controller is configured to lower power to the at least two heating elements under windy conditions.

14. The device of claim 1, wherein for heating arrangement (a), the controller is configured to operate only one of the at least two heating elements for volatizing.

15. In a method of heating a pad with a heating device to volatize compounds impregnated in the pad, the improvement comprising heating the pad using the heating device of claim 1.

16. A heating device comprising:
at least first and second heaters mounted on a substrate in communication with each other;
at least one temperature sensor, optionally more than one temperature sensor;
a controller providing battery power to each of the at least two heating elements;
wherein the controller is configured to power the first heater based on a first temperature, then to power the second heater after the first heating element raises the temperature of both heating elements to a second temperature.

17. The heating device of claim 16 further comprising one or more of:
a) a housing to house the at least first and second heaters, the at least one temperature sensor, and the controller,
b) a battery;
c) a printed circuit board as the at least first and second heaters.

18. The heating device of claim 1, wherein the housing includes a space adjacent to a surface of the substrate that is opposite to a surface of the substrate that faces the at least one opening, the space including insulation therein.

19. The heating device of claim 1, wherein, for the heating arrangement (b), at least two heaters are mounted on the substrate, each controlled individually.

20. The heating device of claim 1, wherein the substrate has a first portion for supporting either the first and second heaters or at least one heater and a second portion supporting the controller, the first and second portions separated by opposing slots forming a connecting portion to minimize heat conduction between the first and second portions.

21. The heating device of claim 1, wherein the housing includes slots sized to receive and hold peripheral ends of the substrate supporting the at least first and second heaters or the at least one heater to minimize heat conduction from the substrate to the housing.

22. The heating device of claim 1, wherein the controller periodically causes power to the device to be on to prevent the battery when having an auto shut off feature from powering down.
